(19) United States
(12) Patent Application Publication
(10) Pub. No.: US 2017/0239614 A1
(43) Pub. Date: Aug. 24, 2017

(54) ENHANCED OLEFIN RECOVERY PROCESS

(71) Applicant: KELLOGG BROWN & ROOT LLC, HOUSTON, TX (US)

(72) Inventors: VIJENDER K. VERMA, SUGAR LAND, TX (US); YANBO LI, Beijing (CN); MICHAEL J. TALLMAN, HOUSTON, TX (US)

(21) Appl. No.: 15/050,251

(22) Filed: Feb. 22, 2016

(51) Int. Cl. B01D 53/18; B01D 53/14; C07C 7/11

(52) U.S. Cl. CPC B01D 53/18; C07C 7/11; B01D 53/1487; B01D 53/1493; B01D 2252/205

(57) ABSTRACT

A demethanizer employing C3, C4 and C4+ hydrocarbons as absorbents can be employed in a methanol to olefin process to reduce both operating and capital costs in comparison to a convention process which employs only cryogenic distillation to remove methane and other low boilers from a light olefin process stream. By reducing the use of propane, the size and operating costs of the C3 splitter can be reduced thereby providing an economic advantage over conventional applications.

### BACKGROUND

1. Field of the Disclosure

This application relates to systems and methods for enhancing recovery of olefins from methanol to olefin processes. This application particularly relates to such processes where the use of propane in the process is mitigated or even eliminated.

2. Description of the Related Art

Olefins are typically produced by converting a hydrocarbon feed at a high temperature to provide a hydrocarbon mixture containing various alkane, alkene, and alkyne hydrocarbons. The hydrocarbon mixture is then fractionated using a series of distillation columns, fractionation columns, compressors, and refrigeration systems to cool, condense, and separate the various hydrocarbon products. In one method of recovering light hydrocarbons, propane is used to absorb light olefins such as ethylene and propylene, and then recycled after separation of the olefin products.

### SUMMARY

In one aspect, the invention is a method for recovering light olefins from a methanol to olefin process stream including using an absorption demethanizer unit, wherein the use of propane as an absorbent is reduced or even eliminated by employing C3 and C4 and heavier compounds and mixtures of C3 and C4 and heavier compounds as the absorbent. By employing the demethanizer unit and using less or even no propane, the capital and operating expenditures for the C3 splitting column is greatly reduced offering a competitive advantage to conventional methods.

It should be understood that examples of certain features of the disclosure have been summarized rather broadly in order that the detailed description thereof that follows may be better understood, and in order that the contributions to the art may be appreciated. There are, of course, additional features of the disclosure that will be described hereinafter and which will in some cases form the subject of the claims appended thereto.

### BRIEF DESCRIPTION OF THE DRAWINGS

For detailed understanding of the present disclosure, references should be made to the following detailed description of the preferred embodiment, taken in conjunction with the accompanying drawings, in which like elements have been given like numerals and wherein:

FIG. 1 depicts a block flow diagram for a demethanizer;

FIG. 2 depicts a block flow diagram for a methanol to olefin process employing mixed C3 and C4+ absorbents; and

FIG. 3 depicts a block flow diagram for a methanol to olefin process employing mixed C3 hydrocarbons and propane as an absorbent in a demethanizer.

### DETAILED DESCRIPTION

In the practice of the method of the application, light olefins are recovered from the effluent of a methanol to olefin process reactor. In such a method, the effluent, after initial treatment to dry and remove undesirable components, becomes a process stream rich in olefins which is then introduced into a demethanizer. The demethanizer employs an absorbent including at least one members selected from the group consisting of C3 hydrocarbons, C3+ hydrocarbons, C4 hydrocarbons, C4+ hydrocarbons, and combinations thereof, with the caveat that when the absorbent is exclusively C3 hydrocarbons, it includes other C3 compounds besides propane.

Absorbents useful with the method of the application include, but are not limited to propylene, one or more butanes, one or more butenes, one or more pentanes, one or more pentenes, and the like. Propane may be used as long as it is not the sole absorbent.

In one aspect, the invention is a method for recovering light olefins from a methanol to olefin process stream including using an absorption demethanizer unit, wherein the use of propane as an absorbent is reduced or even eliminated by employing C3 and C4 and heavier compounds and mixtures of C3 and C4 and heavier compounds as the absorbent. By employing the demethanizer unit and using less or even no propane, the capital and operating expenditures for the C3 splitting column is greatly reduced offering a competitive advantage to conventional methods.

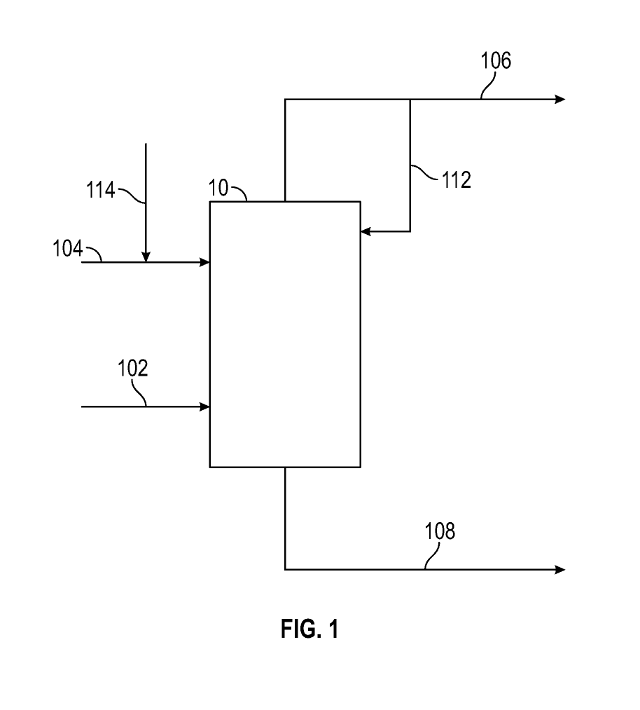

Referring to FIG. 1, therein is shown a system for the extractive distillation unit sometimes also referred in the art as a demethanizer. To avoid prolixity, only elements essential for illustration have been shown. Other devices not illustrated that might be present include but are not limited to pumps, gas compressors, heat exchangers, drums, vessels, reactors, flow lines, valves, and control loops, heat exchange loops and the like. In at least one embodiment of the invention, a light olefin containing stream is supplied to the demethanizer system 10 via flow line 102. In this embodiment, the demethanizer is an absorption column.

The light olefins containing stream enters the demethanizer at a suitable point in the system to effect the desired contact with the C3, C4, and heavier hydrocarbon absorbent fed via flow line 104. For example, the absorbent stream may be fed to the demethanizer at a point above the inlet for stream 102, such that the hydrocarbon solvent flow is countercurrent to the methane, i.e., the solvent flows down the demethanizer to contact the light olefin containing stream counter currently. As the absorbent traverses down the column, the light olefins are absorbed by the absorbent.

The hydrocarbon absorbent and the absorbed light olefins may be recovered from the column as a bottoms fraction via flow line 108. Methane may be recovered from the demethanizer as an overheads fraction via flow line 106. In some embodiments, at least a portion of the overheads fraction may be returned to the demethanizer as a reflux via flow line 112.

In at least some embodiments of the methods of the application, the bottoms fraction will be further treated (not shown in FIG. 1). This further treatment will be by use of conventional cryogenic distillation of the various components, including the C2 and C3 olefin components. Such cryogenic distillation processes include those known to the art, including that disclosed by US 2014/0275674 to Verma et al., which is incorporated herein in its entirety by reference.

By employing the method of the application, the amount of propane used to separate the light olefins from the reactor feed is reduced. Since the C3 fraction includes propylene, a desirable product of methanol to olefin conversion, then reduction of the amount of propane used in the process allows for the reduction in size of the C3 splitter. In embodiments, the amount of propane absorbent can make up less than 50%, 25%, 10%, 5%, or 1% of the total amount of absorbent. In embodiments, no propane absorbent is used. This can be a significant capital expenditure savings during construction.

Taking this a step further, the operation, needing less cryogenic distillation to separate and then recycle propane, can then be operated with a savings of energy. These savings in capital and then operational expenses provides for a substantial competitive advantage over a conventional process.

While the embodiment described above is one including building a new unit, it should be appreciated that the demethanizer may be retrofitted into an existing unit. In an alternative embodiment, a demethanizer from a separate process may be repurposed to do the above described separation and then the effluent returned to an otherwise conventional unit for further processing.

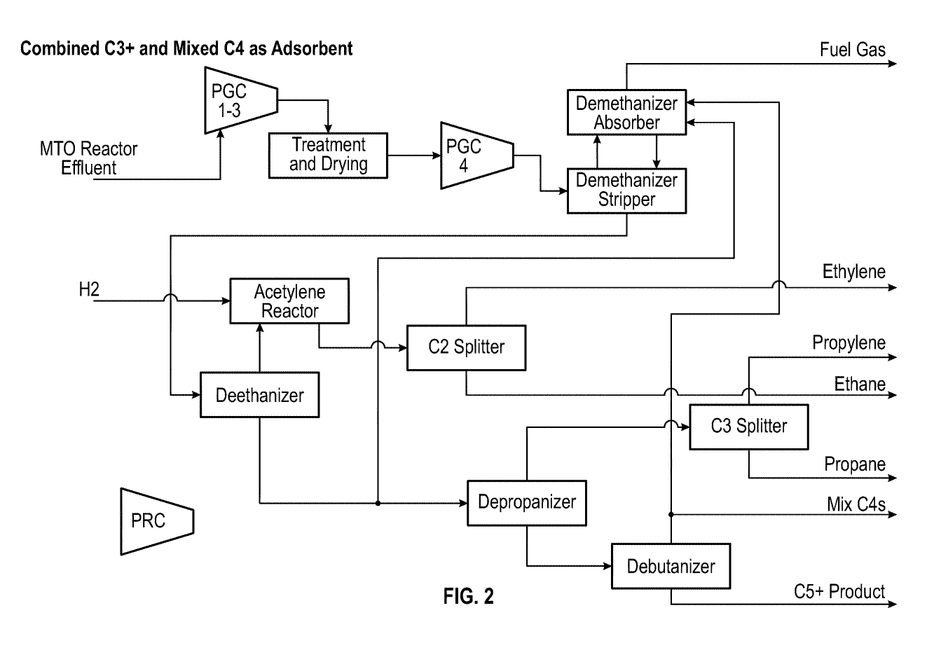

Turning to FIG. 2, a more complete block flow diagram of a process for producing olefins from methanol using combined C3+ and mixed C4 absorbents is shown. A methanol to olefin (MTO) effluent, which may be compressed, treated, and dried, is sent to a compressor and then to a demethanizer stripper and absorber unit. An effluent of the demethanizer unit is sent to a deethanzier. A portion of the effluent from the deethanizer is returned to the demethanizer absorber unit. Another portion of the effluent from the deethanizer is sent to a debutanizer. A portion of the effluent of the debutanizer is sent to the demethanizer absorber unit. Suitable lines, valving, and other flow control mechanisms are used to convey the fluids between the identified equipment and locations.

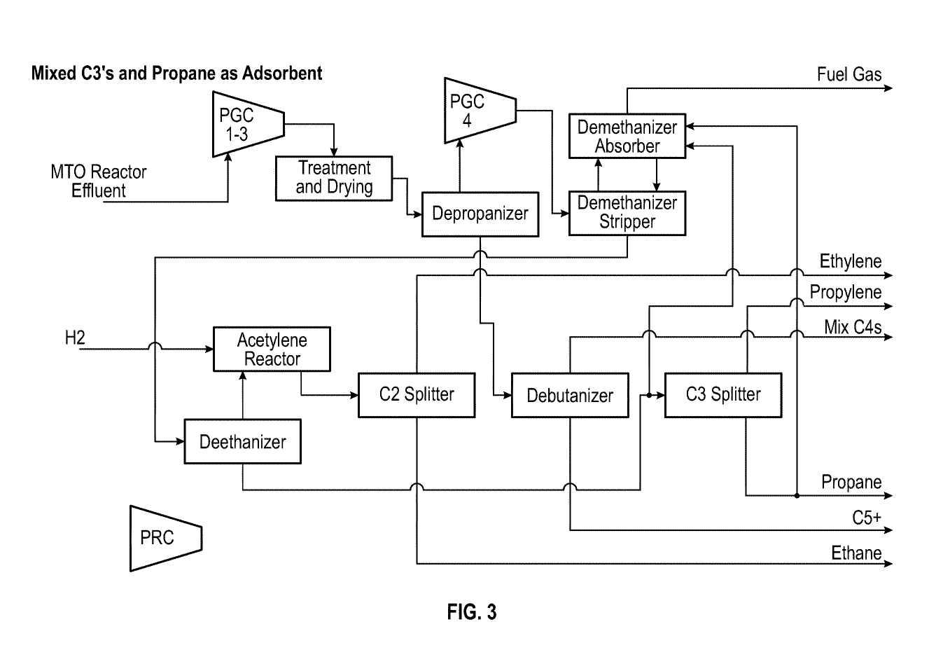

Turning to FIG. 3, a more complete block flow diagram of a process for producing olefins from methanol using mixed C3 and propane absorbents is shown. A methanol to olefin (MTO) effluent, which may be compressed, treated, and dried, is sent to a compressor and then to a demethanizer stripper and absorber unit. An effluent of the demethanizer unit is sent to a deethanizer. A portion of the effluent from the deethanizer is returned to the demethanizer absorber unit. Another portion of the effluent from the deethanizer is sent to a C3 splitter. A portion of the effluent of the C3 splitter is sent to the demethanizer absorber unit. Suitable lines, valving, and other flow control mechanisms are used to convey the fluids between the identified equipment and locations.

While the foregoing is directed to embodiments of the present disclosure, other and further embodiments of the disclosure may be devised without departing from the basic scope thereof, and the scope thereof is determined by the claims that follow.

What is claimed is:

1. A method for producing olefins from methanol, the method comprising employing a demethanizer to remove methane from a light olefin containing process stream wherein the demethanizer uses an absorbent selected from the group consisting of C3, C4, and C4+ hydrocarbons, and combinations thereof, wherein when the absorbent is a C3 hydrocarbon it is not exclusively propane.

2. The method of claim 1, wherein propane makes up less than ten percent of the total amount of the selected absorbent.

3. The method of claim 1, wherein propane makes up less than one percent of the total amount of the selected absorbent.

4. The method of claim 1, wherein an effluent of the demethanizer is sent to a deethanizer.

5. The method of claim 4, wherein a first portion of an effluent from the deethanizer is returned to the demethanizer.

6. The method of claim 5, wherein a second portion of the effluent from the deethanizer is sent to a debutanizer.

7. The method of claim 6, wherein a portion of an effluent of the debutanizer is sent to the demethanizer.

8. The method of claim 5, wherein a third portion of the effluent from the deethanizer is sent to a C3 splitter.

9. The method of claim 8, wherein a portion of an effluent of the C3 splitter is sent to the demethanizer.

10. A system for producing olefins from methanol, comprising:
demethanizer configured to remove methane from a light olefin containing process stream wherein the demethanizer uses an absorbent selected from the group consisting of C3, C4, and C4+ hydrocarbons, and combinations thereof, wherein when the absorbent is a C3 hydrocarbon it is not exclusively propane.

11. The system of claim 10, further comprising a deethanizer receiving an effluent of the demethanizer.

12. The system of claim 10, further comprising a line returning a first portion of an effluent from the deethanizer to the demethanizer.

13. The system of claim 12, further comprising a debutanizer receiving a second portion of the effluent from the deethanizer.

14. The system of claim 13, further comprising a second line sending a portion of an effluent of the debutanizer to the demethanizer.

15. The system of claim 12, further comprising a C3 splitter receiving a third portion of the effluent from the deethanizer.

16. The system of claim 15, further comprising a third line sending a portion of an effluent of the C3 splitter to the demethanizer.
